(19) United States
(12) Patent Application Publication
(10) Pub. No.: US 2019/0020808 A1
(43) Pub. Date: Jan. 17, 2019

(54) REMOTELY CONTROLLABLE CAMERA ON HEAD-MOUNT FOR THE BLIND

(71) Applicant: Sony Corporation, Tokyo (JP)

(72) Inventors: Peter Shintani, San Diego, CA (US); Brant Candelore, Escondido, CA (US); Mahyar Nejat, San Diego, CA (US)

(21) Appl. No.: 15/646,790

(22) Filed: Jul. 11, 2017

(51) Int. Cl. H04N 5/232; G03B 17/56; F16M 13/04; A42B 1/24; A61F 9/08

(52) U.S. Cl. CPC H04N 5/23206; G03B 17/561; H04N 5/23296; A61F 9/08; H04N 5/23216; F16M 13/04; A42B 1/242; H04N 5/23293

(57) ABSTRACT

A camera on a head mount can be remotely controlled by a monitoring device to pan and tilt so that a remote monitor may gain a picture of the surroundings of a vision-impaired person wearing the head mount without pestering the person to move his head.

### FIELD

The present application relates to technically inventive, non-routine solutions that are necessarily rooted in computer technology and that produce concrete technical improvements.

### BACKGROUND

As understood herein, to help a vision-impaired person with daily tasks, remote video monitoring of the person may be used. This may be done by having the person wear “smart” glasses that provide a video feed to a monitor.

### SUMMARY

As understood herein, however, the remote viewer is seeing only where the blind person is pointing his head. As further understood herein, situations may arise in which it would be advantageous to gain a video feed from other directions without making the wearer of the device move his head.

Accordingly, a camera supported on a small rotating gimbal is mounted to a head-wearable mount or cap to allow a remote viewer to pan the camera to the left and right and if desired to tilt it up and down. The camera can be rotated through a complete 360° circle if desired.

In an aspect, an assembly includes a head-wearable support such as a cap that is configured to be worn on a person's head. The assembly includes a camera and a movable coupling connecting the camera to the support. A wireless transceiver is on the support to receive camera motion commands from a monitoring device and to transmit images generated by the camera to the monitoring device. A processor on the support receives images from the camera and send the images through the transceiver to the monitoring device. The processor also moves the coupling to thereby move the camera according to commands received through the transceiver from the monitoring device.

In some embodiments the coupling includes a gimbal. The coupling can be configured to pan the camera azimuthally and/or tilt the camera elevationally. If desired, a speaker such as left and right earbuds can be provided on the apparatus for communicating with the processor.

In another aspect, a monitoring device for a camera on a head-wearable apparatus includes a processor, a display for control by the processor, and storage with instructions executable by the processor to receive from the head-wearable apparatus an image generated by a camera on the head-wearable apparatus. The instructions are executable to present on the display the at least one image. The instructions are further executable to present on the display at least one user interface (UI) with one or more selectors selectable to cause the processor to send a command to the head-wearable apparatus to move the camera.

In another aspect, a method includes receiving video information wirelessly from a head-wearable apparatus, and presenting video based on the video information on a display device remote from the head-wearable apparatus. The method also includes presenting on the display device at least one selector invokable to move a camera on the head-wearable apparatus.

The details of the present disclosure, both as to its structure and operation, can be best understood in reference to the accompanying drawings, in which like reference numerals refer to like parts, and in which:

### BRIEF DESCRIPTION OF THE DRAWINGS

FIG. 1 is a block diagram of an example system including an example in consistent with present principles;

FIG. 2 is a schematic diagram of a vision-impaired person wearing a head mount with a movable camera that communicates with a monitoring device;

FIG. 3 is a block diagram of components of the head mount;

FIG. 4 is a flow chart of logic consistent with present principles; and

FIG. 5 is a screen shot of an example user interface (UI) on a monitoring device consistent with present principles.

### DETAILED DESCRIPTION

This disclosure relates generally to computer ecosystems including aspects of consumer electronics (CE) device based user information in computer ecosystems. A system herein may include server and client components, connected over a network such that data may be exchanged between the client and server components. The client components may include one or more computing devices including portable televisions (e.g. smart TVs, Internet-enabled TVs), portable computers such as laptops and tablet computers, and other mobile devices including smart phones and additional examples discussed below. These client devices may operate with a variety of operating environments. For example, some of the client computers may employ, as examples, operating systems from Microsoft, or a Unix operating system, or operating systems produced by Apple Computer or Google. These operating environments may be used to execute one or more browsing programs, such as a browser made by Microsoft or Google or Mozilla or other browser program that can access web applications hosted by the Internet servers discussed below.

Servers may include one or more processors executing instructions that configure the servers to receive and transmit data over a network such as the Internet. Or, a client and server can be connected over a local intranet or a virtual private network. A server or controller may be instantiated by a game console such as a Sony PlayStation®, a personal computer, etc.

Information may be exchanged over a network between the clients and servers. To this end and for security, servers and/or clients can include firewalls, load balancers, temporary storages, and proxies, and other network infrastructure for reliability and security. One or more servers may form an apparatus that implement methods of providing a secure community such as an online social website to network members.

As used herein, instructions refer to computer-implemented steps for processing information in the system. Instructions can be implemented in software, firmware or hardware and include any type of programmed step undertaken by components of the system.

A processor may be any conventional general purpose single- or multi-chip processor that can execute logic by means of various lines such as address lines, data lines, and control lines and registers and shift registers.

Software modules described by way of the flow charts and user interfaces herein can include various sub-routines, procedures, etc. Without limiting the disclosure, logic stated to be executed by a particular module can be redistributed to other software modules and/or combined together in a single module and/or made available in a shareable library.

Present principles described herein can be implemented as hardware, software, firmware, or combinations thereof; hence, illustrative components, blocks, modules, circuits, and steps are set forth in terms of their functionality.

Further to what has been alluded to above, logical blocks, modules, and circuits described below can be implemented or performed with a general purpose processor, a digital signal processor (DSP), a field programmable gate array (FPGA) or other programmable logic device such as an application specific integrated circuit (ASIC), discrete gate or transistor logic, discrete hardware components, or any combination thereof designed to perform the functions described herein. A processor can be implemented by a controller or state machine or a combination of computing devices.

The functions and methods described below, when implemented in software, can be written in an appropriate language such as but not limited to C# or C++, and can be stored on or transmitted through a computer-readable storage medium such as a random access memory (RAM), read-only memory (ROM), electrically erasable programmable read-only memory (EEPROM), compact disk read-only memory (CD-ROM) or other optical disk storage such as digital versatile disc (DVD), magnetic disk storage or other magnetic storage devices including removable thumb drives, etc. A connection may establish a computer-readable medium. Such connections can include, as examples, hard-wired cables including fiber optics and coaxial wires and digital subscriber line (DSL) and twisted pair wires.

Components included in one embodiment can be used in other embodiments in any appropriate combination. For example, any of the various components described herein and/or depicted in the Figures may be combined, interchanged or excluded from other embodiments.

“A system having at least one of A, B, and C” (likewise “a system having at least one of A, B, or C” and “a system having at least one of A, B, C”) includes systems that have A alone, B alone, C alone, A and B together, A and C together, B and C together, and/or A, B, and C together, etc.

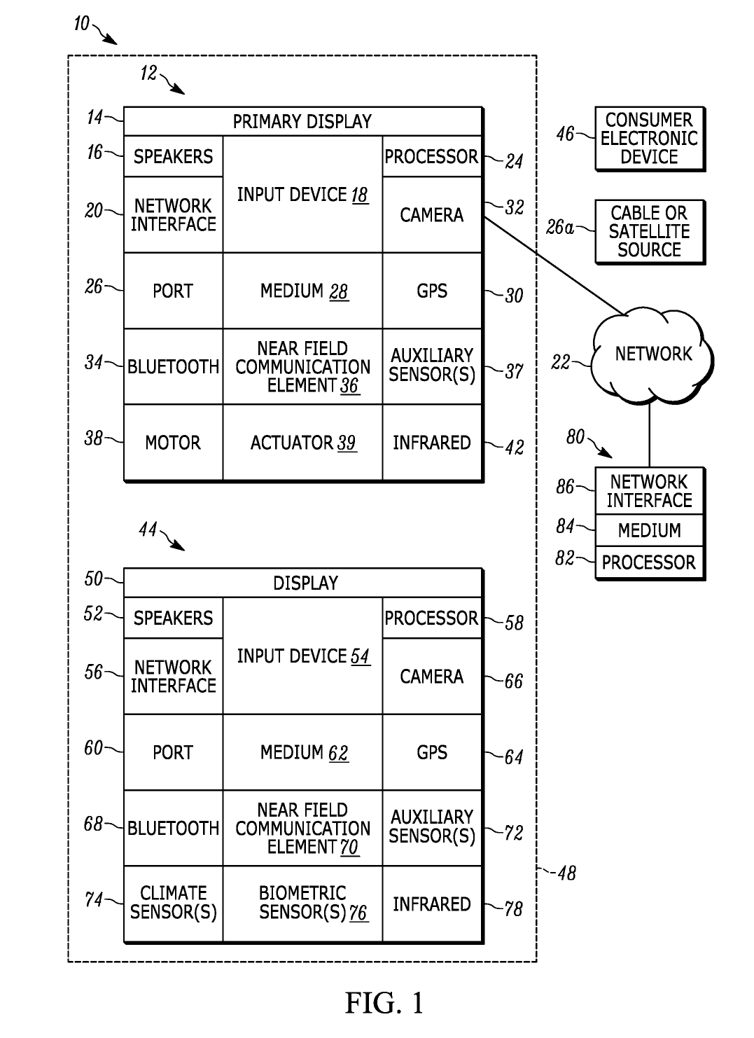

Now specifically referring to FIG. 1, an example ecosystem 10 is shown, which may include one or more of the example devices mentioned above and described further below in accordance with present principles. The first of the example devices included in the system 10 is an example primary display device, and in the embodiment shown is an audio video display device (AVDD) 12 such as but not limited to an Internet-enabled TV. Thus, the AVDD 12 alternatively may be an appliance or household item, e.g. computerized Internet enabled refrigerator, washer, or dryer. The AVDD 12 alternatively may also be a computerized Internet enabled (“smart”) telephone, a tablet computer, a notebook computer, a wearable computerized device such as e.g. computerized Internet-enabled watch, a computerized Internet-enabled bracelet, other computerized Internet-enabled devices, a computerized Internet-enabled music player, computerized Internet-enabled head phones, a computerized Internet-enabled implantable device such as an implantable skin device, etc. Regardless, it is to be understood that the AVDD 12 is configured to undertake present principles (e.g. communicate with other CE devices to undertake present principles, execute the logic described herein, and perform any other functions and/or operations described herein).

Accordingly, to undertake such principles the AVDD 12 can be established by some or all of the components shown in FIG. 1. For example, the AVDD 12 can include one or more displays 14 that may be implemented by a high definition or ultra-high definition “4K” or “8K” (or higher resolution) flat screen and that may be touch-enabled for receiving consumer input signals via touches on the display. The AVDD 12 may include one or more speakers 16 for outputting audio in accordance with present principles, and at least one additional input device 18 such as e.g. an audio receiver/microphone for e.g. entering audible commands to the AVDD 12 to control the AVDD 12. The example AVDD 12 may also include one or more network interfaces 20 for communication over at least one network 22 such as the Internet, an WAN, an LAN, etc. under control of one or more processors 24. Thus, the interface 20 may be, without limitation, a Wi-Fi transceiver, which is an example of a wireless computer network interface. It is to be understood that the processor 24 controls the AVDD 12 to undertake present principles, including the other elements of the AVDD 12 described herein such as e.g. controlling the display 14 to present images thereon and receiving input therefrom. Furthermore, note the network interface 20 may be, e.g., a wired or wireless modem or router, or other appropriate interface such as, e.g., a wireless telephony transceiver, or Wi-Fi transceiver as mentioned above, etc.

In addition to the foregoing, the AVDD 12 may also include one or more input ports 26 such as, e.g., a USB port to physically connect (e.g. using a wired connection) to another CE device and/or a headphone port to connect headphones to the AVDD 12 for presentation of audio from the AVDD 12 to a consumer through the headphones. The AVDD 12 may further include one or more computer memories 28 that are not transitory signals, such as disk-based or solid state storage (including but not limited to flash memory). Also in some embodiments, the AVDD 12 can include a position or location receiver such as but not limited to a cellphone receiver, GPS receiver and/or altimeter 30 that is configured to e.g. receive geographic position information from at least one satellite or cellphone tower and provide the information to the processor 24 and/or determine an altitude at which the AVDD 12 is disposed in conjunction with the processor 24. However, it is to be understood that that another suitable position receiver other than a cellphone receiver, GPS receiver and/or altimeter may be used in accordance with present principles to e.g. determine the location of the AVDD 12 in e.g. all three dimensions.

Continuing the description of the AVDD 12, in some embodiments the AVDD 12 may include one or more cameras 32 that may be, e.g., a thermal imaging camera, a digital camera such as a webcam, and/or a camera integrated into the AVDD 12 and controllable by the processor 24 to gather pictures/images and/or video in accordance with present principles. Also included on the AVDD 12 may be a Bluetooth transceiver 34 and other Near Field Communication (NFC) element 36 for communication with other devices using Bluetooth and/or NFC technology, respectively. An example NFC element can be a radio frequency identification (RFID) element.

Further still, the AVDD 12 may include one or more auxiliary sensors 37 (e.g., a motion sensor such as an accelerometer, gyroscope, cyclometer, or a magnetic sensor, an infrared (IR) sensor, an optical sensor, a speed and/or cadence sensor, a gesture sensor (e.g. for sensing gesture command, etc.) providing input to the processor 24. The AVDD 12 may include still other sensors such as e.g. one or more climate sensors 38 (e.g. barometers, humidity sensors, wind sensors, light sensors, temperature sensors, etc.) and/or one or more biometric sensors 40 providing input to the processor 24. In addition to the foregoing, it is noted that the AVDD 12 may also include an infrared (IR) transmitter and/or IR receiver and/or IR transceiver 42 such as an IR data association (IRDA) device. A battery (not shown) may be provided for powering the AVDD 12.

Still referring to FIG. 1, in addition to the AVDD 12, the system 10 may include one or more other CE device types. In one example, a first CE device 44 may be used to control the display via commands sent through the below-described server while a second CE device 46 may include similar components as the first CE device 44 and hence will not be discussed in detail. In the example shown, only two CE devices 44, 46 are shown, it being understood that fewer or greater devices may be used.

In the example shown, to illustrate present principles all three devices 12, 44, 46 are assumed to be members of an entertainment network in, e.g., in a home, or at least to be present in proximity to each other in a location such as a house. However, for illustrating present principles the first CE device 44 is assumed to be in the same room as the AVDD 12, bounded by walls illustrated by dashed lines 48.

The example non-limiting first CE device 44 may be established by any one of the above-mentioned devices, for example, a portable wireless laptop computer or notebook computer, and accordingly may have one or more of the components described below. The second CE device 46 without limitation may be established by a wireless telephone. The second CE device 46 may implement a portable hand-held remote control (RC).

The first CE device 44 may include one or more displays 50 that may be touch-enabled for receiving consumer input signals via touches on the display. The first CE device 44 may include one or more speakers 52 for outputting audio in accordance with present principles, and at least one additional input device 54 such as e.g. an audio receiver/microphone for e.g. entering audible commands to the first CE device 44 to control the device 44. The example first CE device 44 may also include one or more network interfaces 56 for communication over the network 22 under control of one or more CE device processors 58. Thus, the interface 56 may be, without limitation, a Wi-Fi transceiver, which is an example of a wireless computer network interface. It is to be understood that the processor 58 may control the first CE device 44 to undertake present principles, including the other elements of the first CE device 44 described herein such as e.g. controlling the display 50 to present images thereon and receiving input therefrom. Furthermore, note the network interface 56 may be, e.g., a wired or wireless modem or router, or other appropriate interface such as, e.g., a wireless telephony transceiver, or Wi-Fi transceiver as mentioned above, etc.

In addition to the foregoing, the first CE device 44 may also include one or more input ports 60 such as, e.g., a USB port to physically connect (e.g. using a wired connection) to another CE device and/or a headphone port to connect headphones to the first CE device 44 for presentation of audio from the first CE device 44 to a consumer through the headphones. The first CE device 44 may further include one or more computer memories 62 such as disk-based or solid state storage. Also in some embodiments, the first CE device 44 can include a position or location receiver such as but not limited to a cellphone and/or GPS receiver and/or altimeter 64 that is configured to e.g. receive geographic position information from at least one satellite and/or cell tower, using triangulation, and provide the information to the CE device processor 58 and/or determine an altitude at which the first CE device 44 is disposed in conjunction with the CE device processor 58. However, it is to be understood that that another suitable position receiver other than a cellphone and/or GPS receiver and/or altimeter may be used in accordance with present principles to e.g. determine the location of the first CE device 44 in e.g. all three dimensions.

Continuing the description of the first CE device 44, in some embodiments the first CE device 44 may include one or more cameras 66 that may be, e.g., a thermal imaging camera, a digital camera such as a webcam, and/or a camera integrated into the first CE device 44 and controllable by the CE device processor 58 to gather pictures/images and/or video in accordance with present principles. Also included on the first CE device 44 may be a Bluetooth transceiver 68 and other Near Field Communication (NFC) element 70 for communication with other devices using Bluetooth and/or NFC technology, respectively. An example NFC element can be a radio frequency identification (RFID) element.

Further still, the first CE device 44 may include one or more auxiliary sensors 72 (e.g., a motion sensor such as an accelerometer, gyroscope, cyclometer, or a magnetic sensor, an infrared (IR) sensor, an optical sensor, a speed and/or cadence sensor, a gesture sensor (e.g. for sensing gesture command, etc.) providing input to the CE device processor 58. The first CE device 44 may include still other sensors such as e.g. one or more climate sensors 74 (e.g. barometers, humidity sensors, wind sensors, light sensors, temperature sensors, etc.) and/or one or more biometric sensors 76 providing input to the CE device processor 58. In addition to the foregoing, it is noted that in some embodiments the first CE device 44 may also include an infrared (IR) transmitter and/or IR receiver and/or IR transceiver 78 such as an IR data association (IRDA) device. A battery (not shown) may be provided for powering the first CE device 44.

The second CE device 46 may include some or all of the components shown for the CE device 44.

Now in reference to the afore-mentioned at least one server 80, it includes at least one server processor 82, at least one computer memory 84 such as disk-based or solid state storage, and at least one network interface 86 that, under control of the server processor 82, allows for communication with the other devices of FIG. 1 over the network 22, and indeed may facilitate communication between servers and client devices in accordance with present principles. Note that the network interface 86 may be, e.g., a wired or wireless modem or router, Wi-Fi transceiver, or other appropriate interface such as, e.g., a wireless telephony transceiver.

Accordingly, in some embodiments the server 80 may be an Internet server, and may include and perform “cloud” functions such that the devices of the system 10 may access a “cloud” environment via the server 80 in example embodiments. Or, the server 80 may be implemented by a game console or other computer in the same room as the other devices shown in FIG. 1 or nearby.

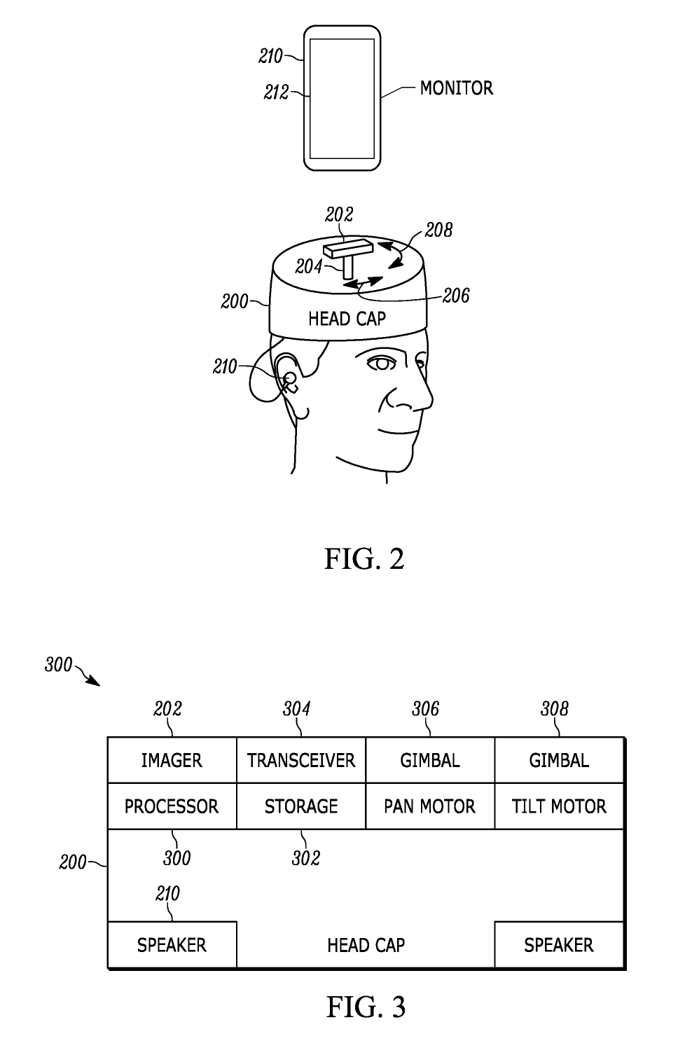

FIG. 2 shows a head-wearable support 200 that as shown is configured to be worn on a person's head. In the example shown, the support 200 is configured as a cap or other hat-type or helmet-type headwear with an interior concave space sized and shaped to fit snugly over a person's head.

As shown in FIG. 2, at least one camera 202 such as a video camera is movably mounted on the support 200 by means of a movable coupling 204 connecting the camera 202 to the support 200. The camera can be panned in the azimuthal dimension (horizontal) as indicated by the arrows 206. If desired, the camera 202 may also be tiled in the elevational dimension that is orthogonal to the azimuthal dimension as indicated by the arrow 208. In some examples, the coupling 204 may include one or more gimbal assemblies that are controlled by the processors described more fully below.

Also, if desired one more speakers such as but not limited to left and right ear buds 210 or earphones may be provided on the support 200 and coupled thereto using flaccid earbud cords, a headset-style rigid arm, and the like.

FIG. 2 also shows that a monitoring device 210 such as but not limited to a wireless communication (WCD) such as a smart phone may communicate with the below-described processor in the support 200 to receive images from the camera 202 and present them on a display 212. It is to be understood that the support 200 may include appropriate components of the first CE device 44 described above whereas the monitoring device 210 may be established by appropriate components of the AVDD 12 or second CE device 46. For example, the monitoring device 210 may include a microphone as shown in FIG. 1 to receive voice signals from a user of the monitoring device 210 and send the voice signals to the head-wearable apparatus for play on the speakers 210.

FIG. 3 illustrates typically internal components of the support 200. As shown, a processor 300 may receive image signals from the camera or other imager 202 and may access computer storage 302 to store the images and to access instructions to execute logic herein. The processor 300 communicates with one or more wireless transceivers 304 to receive camera motion commands from the monitoring device 210 and to transmit images generated by the camera 202 to the monitoring device 210.

The processor 300 also controls one or more camera movable couplings such as gimbals 306, 308 as described above, with the gimbals being coupled to the camera 202 to move the camera. One gimbal may include a pan motor and one gimbal a tilt motor, or a single gimbal that can both tilt and pan may be provided. The processor 300 typically activates the gimbals according to commands received through the transceiver 304 from the monitoring device 210.

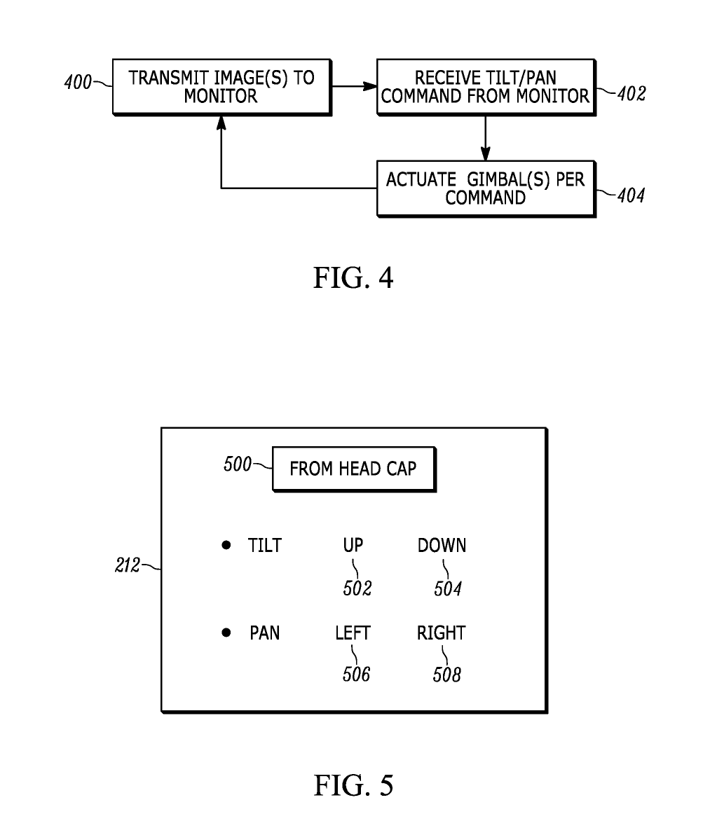

Indeed and referring now to FIGS. 4 and 5, at block 400 of FIG. 4 the processor 300 transmits images from the camera 202 to the monitoring device 210 for presentation of the images 500 (FIG. 5) on the display 212 of the monitoring device.

Also, at block 403 of FIG. 4 the processor 300 of the support 200 may receive camera pan and/or tilt commands from the monitoring device 210, actuating the gimbal(s) 306, 308 at block 404 according to the commands. As shown in FIG. 5, in one non-limiting example a user of the monitoring device 210 may be presented with an up selector 502 to tilt the camera up, a down selector 504 to tilt the camera down, a left selector 506 to pan the camera left, and a right selector 508 to pan the camera right, all directions being relative to a “front” of the support 200 as worn by the person being monitored. It is to be understood that other selector types may be provided and selectable to cause the processor of the monitoring device to send a command to the head-wearable apparatus to move the camera, such as a joystick-style input.

In any case, it may now be appreciated that the monitoring device 210 can receive video information wirelessly from the head-wearable apparatus 200 and present video based on the video information on the display 212, which can be remote from the head-wearable apparatus 200. Selectors invokable to move the camera 202 on the head-wearable apparatus 200 may also be provided on the monitoring device.

In some embodiments, the camera 202 may be provided to the rear of the head-mounted apparatus 200. The remote camera is also useful for a monitoring child.

While particular techniques are herein shown and described in detail, it is to be understood that the subject matter which is encompassed by the present application is limited only by the claims.

What is claimed is:

1. Assembly comprising:
a head-wearable support configured to be worn on a person's head;
at least one camera;
at least one movable coupling connecting the camera to the support;
at least one wireless transceiver on the support to receive camera motion commands from a monitoring device and transmit images generated by the camera to the monitoring device; and
at least one processor on the support to receive images from the camera and send the images through the transceiver to the monitoring device, and to move the coupling to thereby move the camera according to commands received through the transceiver from the monitoring device.

2. The assembly of claim 1, wherein the coupling comprises at least one gimbal.

3. The assembly of claim 1, wherein the coupling is configured to pan the camera azimuthally and tilt the camera elevationally.

4. The assembly of claim 1, comprising at least one speaker communicating with the processor.

5. The assembly of claim 4, wherein the at least one speaker comprises left and right ear buds.

6. The assembly of claim 1, wherein the coupling is configured to pan the camera azimuthally.

7. The assembly of claim 1, wherein the coupling is configured to tilt the camera elevationally.

8. The assembly of claim 1, wherein the head-wearable support is configured as a cap for fitting over a person's head.

9. A monitoring device for a camera on a head-wearable apparatus, comprising:
at least one processor;
at least one display for control by the processor; and
at least one storage with instructions executable by the processor to:
receive from the head-wearable apparatus at least one image generated by a camera on the head-wearable apparatus;
present on the display the at least one image; and
present on the display at least one user interface (UI) comprising at least one selector selectable to cause the processor to send a command to the head-wearable apparatus to move the camera.

10. The apparatus of claim 9, wherein the command is a pan command.

11. The apparatus of claim 9, wherein the command is a tilt command.

12. The apparatus of claim 9, comprising at least one microphone, wherein the instructions are executable to:
send voice signals from the at least one microphone to the head-wearable apparatus.

13. A method comprising:
receiving video information wirelessly from a head-wearable apparatus;
presenting video based on the video information on a display device remote from the head-wearable apparatus; and
presenting on the display device at least one selector invokable to move a camera on the head-wearable apparatus.

14. The method of claim 13, wherein the at least one selector is invokable to pan the camera.

15. The method of claim 13, wherein the at least one selector is invokable to tilt the camera.

16. The method of claim 13, wherein the at least one selector is invokable to pan and tilt the camera.

17. The method of claim 13, comprising:
receiving voice signals from a microphone of the display device; and
wirelessly transmitting the voice signals to the head-wearable apparatus.
